SEQUENCE LISTING (1 sequence)

SEQ ID NO: 1 - DNA, 29903 bp, Severe acute respiratory syndrome coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

(19) United States
(12) Patent Application Publication
(10) Pub. No.: US 2023/0270792 A1
(43) Pub. Date: Aug. 31, 2023

(54) REDUCTION OF COVID-19 COAGULOPATHY AND OTHER INFLAMMATION-ASSOCIATED COAGULOPATHIES BY ADMINISTRATION OF FIBROBLASTS

(71) Applicant: FIGENE, LLC, Houston, TX (US)

(72) Inventors: Pete O'HEERON, Houston, TX (US); Thomas ICHIM, San Diego, CA (US)

(21) Appl. No.: 18/040,345

(22) PCT Filed: Aug. 11, 2021
(86) PCT No.: PCT/US2021/071163

§ 371 Date: Feb. 2, 2023

Related U.S. Application Data
(60) Provisional application No. 63/064,538, filed on Aug. 12, 2020.

(51) Int. Cl. A61K 35/33; C12N 5/077

(52) U.S. Cl. CPC A61K 35/33; C12N 5/0656; C12N 2501/31; C12N 2501/148; C12N 2501/20; C12N 2501/113; C12N 2501/165

(57) ABSTRACT

Embodiments of the disclosure include methods and compositions for treatment or reduction in risk of coagulopathy of any kind, including associated with inflammation. In specific embodiments, the coagulopathy is associated with upregulated production of tissue factor in the individual. In specific embodiments, the coagulopathy is in an individual that has SARS-CoV-2 infection or is at risk for having SARS-CoV-2 infection. In specific embodiments, the methods and compositions include fibroblasts, and/or modified fibroblasts, and/or derivatives of fibroblasts, including those fibroblasts exposed to TNF-alpha or one or more other inflammatory agents before activation of the fibroblasts.

### CROSS-REFERENCE TO RELATED APPLICATIONS

This application claims priority to U.S. Provisional Patent Application Ser. No. 63/064,538, filed Aug. 12, 2020, which is incorporated by reference herein in its entirety.

### TECHNICAL FIELD

Embodiments of the disclosure encompass at least the fields of cell biology, molecular biology, immunology, and medicine.

### BACKGROUND

The extremely contagious novel type of beta coronavirus, SARS-CoV-2 (severe acute respiratory syndrome coronavirus-2; previously known as 2019-nCoV), is spreading rapidly around the world, causing a sharp rise of a pneumonia-like illness termed Coronavirus Disease 2019 (COVID-19) [1, 2]. COVID-19 presents a high mortality rate, estimated at 3.4% by the World Health Organization [3]. The rapid spread of the virus (estimated reproductive number R₀ 2.2-3.6) [4, 5] is causing a significant surge of patients requiring intensive care. More than 1 out of 4 hospitalized COVID-19 patients have required admission to an Intensive Care Unit (ICU) for respiratory support, and a large proportion of these ICU-COVID-19 patients, between 17% and 46%, have died [6-10].

A common observation among patients with severe COVID-19 infection is systemic inflammatory response, as well as localization of inflammation to the lower respiratory tract [11-13]. This inflammation, associated with dyspnea and hypoxemia, in some cases evolves into excessive immune response with cytokine storm, determining progression to Acute Lung Injury (ALI), Acute Respiratory Distress Syndrome (ARDS), organ failure, and death [2, 10]. Draconian measures have been put in place in an attempt to curtail the impact of the COVID-19 epidemic on population health and healthcare systems. However, WHO has now classified COVID-19 a pandemic [3]. Crucially, no options are available for those patients with rapidly progressing ARDS evolving to organ failure. Although supportive care is provided whenever possible, including mechanical ventilation and support of vital organ functions, it is insufficient in most severe cases. Therefore, there is an urgent need for novel approaches that can be deployed in a rapid manner to reduce infectivity and/or prevent progression to advanced COVID-19.

In the beginning of the pandemic, much attention has been paid to the pulmonary epithelium as the major area of SARS-CoV-2 infection, in part due to the pulmonary consequences of infection, as well as high expression of ACE2 on type 2 pulmonary epithelial cells. As the pandemic continued, two peculiar aspects of the disease started becoming apparent: the first was that the pulmonary failure seen in COVID-19 patients has characteristics different that conventional ARDS; particularly, there was increased pulmonary compliance in COVID-19 associated cases [14]. Patients presented pulmonary hypoxic vasoconstriction, being associated with severe hypoxemia with “normal” (>40 mLcmH2O-1) lung compliance and likely representing pulmonary microvascular thrombosis [15]. There are cases in which the lung functions appear normal to mildly deficient, but the patient oxygenation levels are exceptionally low. Some have called this state “happy hypoxia” and have speculated possible neurological causes [16-19]. The other abnormality is the systemic coagulation that seems to be occurring all over the body, however, it has some aspects different than the conventional disseminated intravascular coagulation (DIC) that is seen in sepsis. The main difference is that in DIC platelets are consumed leading to thrombocytopenia, and microangiopathy is not seen. In contrast, in COVID-19 coagulopathy, mild if any thrombocytopenia is observed and micro- and macroangiopathy is seen [20]. Additionally, in COVID-19 associated coagulopathy, increased fibrinogen is observed, whereas in DIC, levels of fibrinogen are decreased [21].

It is becoming increasingly accepted that the abnormal and COVID-19 unique coagulation state in certain patients contributes to morbidity and mortality. This type of coagulopathy has been termed by some as “COVID19 Associated Coagulopathy” (CAC) [22-24]. For example, in one study, 388 patients were examined in which thromboembolic events occurred in 28 patients with pulmonary embolism confirmed in 10 patients, with other patients having ischemic stroke and cardiac emboli. The authors noted that the high number of arterial and venous thromboembolic events diagnosed within 24 h of admission suggests there is a previously unrecognized aspect of COVID-19 pathology associated a hypercoagulation state that is different than conventional DIC [25]. In another study, 117 COVID-19 patients were examined. Of the patients who were admitted to ICU, 42% had CAC, while 16% did not. Furthermore, only 2% of patients without CAC required invasive mechanical ventilation, whereas 21% of patients with CAC did [24]. This study, as well as numerous others, support the consideration that in COVID-19, pathology is not simply cytokine release syndrome causing classical ARDS, but pathology is associated with a more insidious, systemic coagulation that causes widespread organ damage and inflammation. It is generally believed that 50% of advanced COVID19 patients have some sort of coagulopathy. This coagulopathy is manifested in at least 40% of patients having pulmonary embolisms and/or DVT despite being on standard doses of anticoagulants such as low molecular weight heparin [26]. Initiation of coagulation could come from various sources. In some studies, it has been shown that direct viral infection of pulmonary epithelium can trigger expression of tissue factor, which initiates the extrinsic pathway of blood coagulation [27]. Importantly, examination of autopsies revealed widespread microthrombi disseminated throughout the pulmonary vasculature, suggesting that vasculopathy/thrombosis is important in COVID-19 pathogenesis [28].

The endothelium was once through to be an inert biological barrier whose function is to maintain stability and integrity of the blood vessel. It was later found that the endothelium plays numerous active processes depending on needs of the body, including modulation of angiogenesis, hemostasis, and immune functions. The endothelial cells are known to be overly sensitive to inflammation. This is believed to be due to the fact that when immune cells are needed in a certain anatomical location, the endothelium upregulates expression of various adhesion molecules, which allow for recruitment of immune cells [29-31]. Additionally, the endothelium controls migration and extravasation of regenerative cells such as stem cells in response to injury [32-34].

When one assesses the endothelium of patients with COVID-19 there are numerous indications to suggest an activated and/or damaged phenotype. In one study, the marker of endothelial cell activation, soluble von Willebrand factor (VWF) was assessed in plasma of 48 COVID-19 patients admitted to the ICU vs in 20 non-ICU patients, elevation compared to normal was 565% and 278% respectively. The paper also reported that plasma concentrations of P-selectin another marker of endothelial activation is also significantly elevated in patients with COVID-19 who are admitted to the ICU vs non-ICU patients [35]. Increases of soluble VWF and P-selectin have been reported by several other studies in COVID-19 patients and correlates with poor prognosis [21, 36-42]. Importantly, blockade of P-Selectin binding to its ligand has been reported to reduce pathology in models of COVID-19 [43]. VWF is a protein which acts to induce platelet adhesion to each other and to blood vessel walls via binding to exposed subendothelial collagen. The actions of VWF is to form platelets clots which plug holes in blood vessel walls to help stop bleeding. VWF increases clotting through binding to and prolonging the half-life of factor VIII. The association between VWF and inflammation is observed in many other inflammatory conditions such as vasculitis [44], aging [45], and diabetes [46, 47].

Endothelial inflammation may also be associated with viral infection. It has been shown that SARS-CoV-2 can directly enter endothelial cells through the ACE2 protein [48]. It is accepted that ACE2 is expressed in human vascular endothelium, respiratory epithelium, and other cell types, and is thought to be a primary mechanism of SARS-CoV-2 entry and infection. In regulator physiological situations, ACE2 acts via its carboxypeptidase activity to generate angiotensin fragments (Ang 1-9 and Ang 1-7), and plays an essential role in the renin-angiotensin system (RAS), which is a critical regulator of cardiovascular homeostasis. SARS-CoV-2 via its surface spike glycoprotein interacts with ACE2 and invades the host cells. Once inside the host cells, SARS-CoV-2 induces acute respiratory distress syndrome (ARDS), stimulates immune response (i.e., cytokine storm) and vascular damage. SARS-CoV-2 induced endothelial cell injury could exacerbate endothelial dysfunction, which is a hallmark of aging, hypertension, and obesity, leading to further complications [49]. By binding to ACE2, downregulation of ACE2 activity occurs as a result of the infection. Reduced ACE2 results in increase angiotensin 2. It is known that augmentation of angiotensin II causes endothelial activation and inflammation. This inflammation may be what is causing damage, in specific embodiments of the disclosure. For example, in one study it was demonstrated that infusion of angiotensin II results in enhanced endothelial expression of adhesion molecules and upregulation of the prototypic inflammatory receptor TLR4 [50]. Upregulation of TLR4 by angiotensin II has been described by numerous other studies that reveal this to be a mechanism of augmented endothelial activation, as well as increased propensity to attract leukocytes [51-54].

### BRIEF SUMMARY

Embodiments of the disclosure include methods and compositions related to treatment, prevention, or reduction in the risk for coagulopathy of any kind, including associated with a viral infection of any kind (such as SARS-CoV-2). In specific embodiments, the disclosure concerns methods of reducing coagulopathy or reducing the risk of coagulopathy by administering to an individual in need thereof a therapeutically effective amount of fibroblasts, and/or modified fibroblasts, and/or derivatives of fibroblasts. In specific cases, the coagulopathy is associated with upregulated production of tissue factor in the individual. The tissue factor may be expressed on endothelial cells and/or monocytes. The coagulopathy may be associated with reduction of one or more endothelial anticlotting factors, such as Protein C, thrombomodulin, and/or anti-thrombin III. One or more anticlotting factors are expressed on endothelial cells and/or on monocytes. The coagulopathy may be caused by or is associated with inflammation. The inflammation may be associated with a 50% or more increase in plasma TNF-alpha, interleukin (IL)-1, IL-6, IL-8, IL-12, IL-17, IL-18, IL-21, IL-27, IL-33, interferon gamma, concentration as compared to an age-matched healthy control subject.

In specific embodiments the fibroblasts are derived from a specific source, such as derived from a tissue selected from the group consisting of a) placenta; b) skin; c) Wharton's Jelly; d) adipose; e) bone marrow; f) peripheral blood; g) cord blood; h) omentum; i) amniotic fluid; j) amniotic membrane; or k) a combination thereof. The fibroblasts, and/or modified fibroblasts, and/or derivatives of fibroblasts may be autologous, allogeneic, or xenogeneic with respect to the individual. In specific embodiments, the fibroblasts proliferate at a rate of 14-21 hours per cell multiplication. The fibroblasts may secrete 0.1 pg-100 pg of interleukin 1 receptor antagonist per culture of 1 million fibroblasts on a 75% confluent surface. The fibroblasts may secrete 0.1 pg-10 pg of interleukin 1 receptor antagonist per culture of 1 million fibroblasts on a 75% confluent surface. The fibroblasts may secrete 1 pg-500 pg of FGF-1 per culture of 1 million fibroblasts on a 75% confluent surface. In some embodiments, the fibroblasts substantially decrease the ability of responding T cells to proliferate in a mixed lymphocyte reaction. A substantial decrease in proliferation may constitute a decrease of more than 20% as compared to a control mixed lymphocyte reaction in which fibroblasts are not added. In specific embodiments, the fibroblasts are treated with an effective amount of hCG to augment immune modulatory activity, such as hCG being administered to the fibroblasts at a concentration of 1 nano Molar to 1 micro Molar per 1 million fibroblasts. The hCG may be administered to the fibroblasts at a concentration of 10 nano Molar to 100 nano Molar per 1 million fibroblasts. In specific aspects, the fibroblasts are treated with TNF-alpha for a sufficient length of time and concentration to allow said fibroblasts to decrease expression of tissue factor on monocytes and/or endothelial cells. The fibroblasts may be treated with 0.1 pg to 20 ng of TNF-alpha per million fibroblast cells for the time period of at least one second. In cases wherein fibroblast derivatives are utilized, the fibroblast derivative may comprise conditioned media from fibroblast culture, microvesicles obtained from fibroblasts, fragments of fibroblasts, exosomes obtained from fibroblasts, apoptotic vesicles obtained from fibroblasts, or a combination thereof. The fibroblasts may be activated with one or more cytokines, one or more growth factors, or a mixture thereof. Cytokines may be selected from the group consisting of IFN-gamma, TNF-alpha, interleukin(IL)-1, IL-6, IL-7, IL-8, IL-12, IL-15, IL-17, IL-33, and a combination thereof. Growth factors may be selected from the group consisting of FGF-1, VEGF, and a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the FIGURES is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIGS. 1A and 1B. FIG. 1A shows reduction of tissue factor expression in monocytes. FIG. 1B shows reduction of tissue factor expression in endothelial cells.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. In specific embodiments, aspects of the disclosure may “consist essentially of” or “consist of” one or more sequences of the disclosure, for example. Some embodiments of the invention may consist of or consist essentially of one or more elements, method steps, and/or methods of the disclosure. It is contemplated that any method or composition described herein can be implemented with respect to any other method or composition described herein. The scope of the present application is not intended to be limited to the particular embodiments of the process, machine, manufacture, composition of matter, means, methods and steps described in the specification. As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The term “coagulopathy” as used herein refers to a dysfunction of hemostasis resulting in either excessive bleeding or clotting, or both.

The term “derivative of fibroblasts” (also “fibroblast-associated product”), as used herein, refers to a molecular or cellular agent derived or obtained from one or more fibroblasts. In some cases, a derivative of fibroblasts is a molecular agent. Examples of molecular derivatives of fibroblasts include conditioned media from fibroblast culture, microvesicles obtained from fibroblasts, fragments of fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and/or lipids obtained from fibroblasts. In some cases, a derivative of fibroblasts is a cellular agent. Examples of cellular derivatives of fibroblasts include cells (e.g., stem cells, hematopoietic cells, neural cells, etc.) produced by differentiation and/or de-differentiation of fibroblasts.

The term “subject” or “patient” or “individual” refer to either a human or non-human, such as primates, mammals, and vertebrates. In particular embodiments, the subject is a human. The subject is of any age, gender, or race.

The term “treatment” refers to a therapeutic intervention that ameliorates a sign or symptom of a disease or pathological condition, such a sign or symptom related to viral infection or prevention. In particular examples, treatment includes preventing a viral infection, for example by inhibiting the full development of a disease or condition associated with the virus. Prevention of a disease does not require a total absence of disease. For example, a decrease of at least 50% can be sufficient. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” or “effective” as used throughout this application refers to anything that promotes or enhances the well-being of the subject with respect to the medical treatment of this condition. This includes, but is not limited to, a reduction in the frequency or severity of the signs or symptoms of a viral infection and associated disease or medical condition.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

### I. Embodiments of the Methods

Disclosed are means, methods, and compositions of matter useful for the prevention of, and/or treatment, of coagulopathy of any kind, including that associated with inflammation of any kind. In some cases, the individual with the coagulopathy may have excessive clotting or clotting that indirectly leads to excessive bleeding. The prevention and/or treatment is addressed by administering an effective amount of fibroblasts, and/or modified fibroblasts, and/or derivatives of fibroblasts to the individual. The methods encompass reducing the risk of coagulopathy in individuals at risk thereof, and also encompass reducing the severity of the coagulopathy and/or delaying the onset of the coagulopathy.

In some embodiments, the disclosure encompasses reduction of or reduced risk of coagulopathy (at least in part) caused by any viral infection, and in further embodiments the disclosure encompasses reduction of coagulopathy caused by SARS-CoV-2 (COVID-19). In certain cases, an individual that tests positive for SARS-CoV-2 is administered an effective amount of fibroblasts, and/or modified fibroblasts, and/or derivatives of fibroblasts, and this reduces the severity of the infection, such as resulting in no or fewer symptom(s) and/or less severe symptom(s). In some cases an individual has not tested positive or has not been tested and is administered an effective amount of fibroblasts, and/or modified fibroblasts, and/or derivatives of fibroblasts, and this reduces the likelihood of the infection and/or delay in onset of infection. In some embodiments, an individual that has been given a vaccine for SARS-CoV-2 is administered an effective amount of fibroblasts, and/or modified fibroblasts, and/or derivatives of fibroblasts, and this reduces the likelihood of an infection or if the individual later becomes infected with SARS-CoV-2 it reduces the severity of the infection, such as resulting in no or fewer symptom(s) and/or less severe symptom(s) and/or delay in onset of infection. In some embodiments, an individual has not been given a vaccine for SARS-CoV-2 is administered an effective amount of fibroblasts, and/or modified fibroblasts, and/or derivatives of fibroblasts, and this reduces the likelihood of an infection or if the individual later becomes infected with SARS-CoV-2 it reduces the severity of the infection, such as resulting in no or fewer symptom(s) and/or less severe symptom(s) and/or delay in onset of infection.

In particular embodiments, the disclosure provides methods for suppressing expression of one or more coagulation-promoting proteins and/or provides methods for enhancing expression of one or more anticoagulation proteins by co-culture or administration of fibroblasts. In one embodiment of the disclosure, there is suppression of tissue factor expression on endothelial and/or monocytic cells by fibroblasts and/or modified fibroblasts and/or derivatives of fibroblasts. In another embodiment, the disclosure provides that fibroblasts and/or modified fibroblasts and/or derivatives of fibroblasts can enhance expression of one or more clot-inhibiting proteins on monocytes and/or endothelial cells, such as thrombomodulin, protein C, and/or anti-thrombin III, etc.

In particular embodiments, the coagulopathy is associated with tissue factor expression on endothelial and/or monocytic cells, including excessive levels of expression compared to an individual lacking coagulopathy. Tissue factor, also called platelet tissue factor, factor III, or CD142, is a protein encoded by the F3 gene that is present in subendothelial tissue and leukocytes. Its role in the clotting process includes the initiation of thrombin formation from the zymogen prothrombin. One example of tissue factor is the protein at GenBank® Accession No. NP_001171567 or the nucleic acid at GenBank® Accession No. NM_001993.

Embodiments of the disclosure include methods and compositions for reducing the level of tissue factor in an individual by administering to the individual an effective amount of fibroblasts and/or modified fibroblasts and/or derivatives of fibroblasts, including for the specific purpose of reducing the level. The individual may or may not be infected with SARS-CoV-2 and the individual may be at risk for infection with SARS-CoV-2. The individual may or may not be vaccinated for SARS-CoV-2.

In some embodiments, methods of the disclosure encompass methods of increasing levels of one or more clot-inhibiting proteins on monocytes and/or endothelial cells, such as increasing expression of thrombomodulin, protein C, and/or antithrombin III. Such methods encompass administering to the individual an effective amount of fibroblasts and/or modified fibroblasts and/or derivatives of fibroblasts, including for the specific purpose of increasing levels of one or more clot-inhibiting proteins on monocytes and/or endothelial cells, such as increasing expression of thrombomodulin, protein C, and/or antithrombin III. The individual may or may not be infected with SARS-CoV-2 and the individual may be at risk for infection with SARS-CoV-2. The individual may or may not be vaccinated for SARS-CoV-2.

An individual at risk for infection with SARS-CoV-2 may be an unvaccinated individual or an individual with a co-morbidity, such as obesity, diabetes, the elderly, chronic kidney disease, any chronic lung disease, heart disease, cancer, chronic liver disease, Immunocompromised state, HIV infection, or a combination thereof. The individual at risk may be at risk when compared to the general population or when compared to one or more individuals that lack one or more (or other) of the examples of co-morbidities.

In certain embodiments of the disclosure, the methods include one or more steps for identifying whether (or that) the individual has coagulopathy. Examples of tests that may be utilized to determine whether an individual has coagulopathy include at least complete blood count, Factor V assay, fibrinogen level, prothrombin time, platelet count, thrombin time, bleeding time, or a combination thereof. Any individual being treated by methods of the disclosure may or may not have ARDS and/or may or may not have cytokine storm.

Embodiments of the disclosure include the aforementioned methods utilizing effective amounts of fibroblasts and/or modified fibroblasts and/or derivatives of fibroblasts.

### II. Fibroblasts, and/or Modified Fibroblasts, and/or Derivatives of Fibroblasts

In some embodiments, the methods encompass administration of an effective amount of fibroblasts and/or modified fibroblasts and/or derivatives of fibroblasts. Thus, in some embodiments the methods utilize fibroblasts that are not modified because they have not been exposed to one or more agents that activate fibroblasts. In other embodiments the methods utilize fibroblasts that are modified because they have been exposed to one or more agents that activate fibroblasts. In some cases a mixture of activated and non-activated fibroblasts may be utilized. In any of these methods, instead of non-activated and/or activated fibroblasts being utilized, derivatives of fibroblasts (or mixtures with non-activated and/or activated fibroblasts) may be utilized.

In some embodiments, prior to use fibroblasts are pre-activated (for example, in culture) with one or more agents before activation. In specific embodiments, the one or more agents are from immune cells (e.g., peripheral blood mononuclear cells (PBMCs), monocytes, monocyte progenitor cells, lymphocytes, macrophages, or a mixture thereof). In particular embodiments, the one or more agents comprise one or more cytokines, one or more growth factors, or a mixture thereof. Examples of cytokines include those selected from the group consisting of IFN-gamma, TNF-alpha, interleukin(IL)-1, IL-6, IL-7, IL-8, IL-12, IL-15, IL-17, IL-33, and a combination thereof. In specific cases, TNF-alpha or one or more other inflammatory agents are exposed to fibroblasts before activation. In specific cases, the growth factor is selected from the group consisting of FGF-1, VEGF, and a combination thereof.

In specific embodiments, the fibroblasts and/or modified fibroblasts and/or derivatives of fibroblasts are exposed, such as in culture, to one or more additional conditions comprises exposure to a media, such as Roswell Park Memorial Institute (RPMI-1640), Dublecco's Modified Essential Media (DMEM), Eagle's Modified Essential Media (EMEM), Optimem, Iscove's Media, or combinations thereof. The fibroblasts and/or modified fibroblasts and/or derivatives of fibroblasts may be exposed, such as in culture, to human platelet rich plasma, platelet lysate, umbilical cord blood serum, autologous serum, human serum, serum replacement, or a combination thereof.

In one embodiment of the disclosure, fibroblasts and/or modified fibroblasts and/or derivatives of fibroblasts are administered to a subject by any suitable route, including by injection (such as intramuscular injection), including in hypoxic areas. Suitable routes include intravenous, subcutaneous, intrathecal, oral, intrarectal, intrathecal, intra-omentral, intraventricular, intrahepatic, and intrarenal.

In certain embodiments, fibroblasts may be derived from tissues comprising skin, heart, blood vessels, bone marrow, skeletal muscle, liver, pancreas, brain, adipose tissue, foreskin, placental, and/or umbilical cord. In specific embodiments, the fibroblasts are placental, fetal, neonatal or adult or mixtures thereof.

The number of administrations of cells to an individual will depend upon the factors described herein at least in part and may be optimized using routine methods in the art. In specific embodiments, a single administration is required. In other embodiments, a plurality of administration of cells is required. It should be appreciated that the system is subject to variables, such as the particular need of the individual, which may vary with time and circumstances, the rate of loss of the cellular activity as a result of loss of cells or activity of individual cells, and the like. Therefore, it is expected that each individual could be monitored for the proper dosage, and such practices of monitoring an individual are routine in the art.

In some embodiments, fibroblasts are utilized in an autologous manner. In another embodiment allogeneic fibroblasts are utilized for the practice of the methods of the disclosure. Various sources of fibroblasts may be used for the practice of the invention, these include: a) foreskin; b) adipose tissue; c) skin biopsy; d) bone marrow; e) placenta; f) umbilical cord; g) amniotic fluid; h) umbilical cord blood; i) ear lobe skin; j) embryonic fibroblasts; k) plastic surgery related by-product; and 1) nail matrix.

In one embodiment, the disclosure encompasses the use of activation of fibroblasts prior to therapeutic use, and/or administration of one or more agents that act upon the fibroblasts, such as act as “regenerative adjuvants” for said fibroblasts. The activated fibroblast cells in the formulation display typical fibroblast morphologies when growing in cultured monolayers. Specifically, cells may display an elongated, fusiform or spindle appearance with slender extensions, or cells may appear as larger, flattened stellate cells that may have cytoplasmic leading edges. A mixture of these morphologies may also be observed. The cells express proteins characteristic of normal fibroblasts including the fibroblast-specific marker, CD90 (Thy-1), a 35 kDa cell-surface glycoprotein, and the extracellular matrix protein, collagen. In one example, the fibroblast dosage formulation may be an autologous cell therapy product comprised of a suspension of autologous fibroblasts, such as grown from a biopsy of each individual's own skin using standard tissue culture procedures. In one embodiment the fibroblasts of the disclosure can also be used to create other cell types for tissue repair or regeneration.

In a particular embodiment, when fibroblasts are administered to the individual, there may be a certain amount of the cells administered. For example, about 50 million to 500 million fibroblast cells are administered to the subject. For example, about 50 million to about 100 million fibroblast cells, about 50 million to about 200 million fibroblast cells, about 50 million to about 300 million fibroblast cells, about 50 million to about 400 million fibroblast cells, about 100 million to about 200 million fibroblast cells, about 100 million to about 300 million fibroblast cells, about 100 million to about 400 million fibroblast cells, about 100 million to about 500 million fibroblast cells, about 200 million to about 300 million fibroblast cells, about 200 million to about 400 million fibroblast cells, about 200 million to about 500 million fibroblast cells, about 300 million to about 400 million fibroblast cells, about 300 million to about 500 million fibroblast cells, about 400 million to about 500 million fibroblast cells, about 50 million fibroblast cells, about 100 million fibroblast cells, about 150 million fibroblast cells, about 200 million fibroblast cells, about 250 million fibroblast cells, about 300 million fibroblast cells, about 350 million fibroblast cells, about 400 million fibroblast cells, about 450 million fibroblast cells or about 500 million fibroblast cells may be administered to the subject.

In a specific embodiment, the fibroblasts utilized in the disclosure may be generated, in one embodiment, by outgrowth from a biopsy of the recipient's own skin (in the case of autologous preparations), or skin of healthy donors (for allogeneic preparations). In some embodiments, fibroblasts are used from young donors. In another embodiment fibroblasts are transfected with one or more genes to allow for enhanced growth and overcoming of the Hayflick limit. Subsequent to derivation the cells may be expanded in culture using standard cell culture techniques. In one specific case, skin tissue (dermis and/or epidermis layers) may be biopsied from a subject's post-auricular area. In one embodiment, the starting material is comprised of multiple (e.g., 2, 3, 4, 5, 10, etc.) 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility. In one embodiment, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany). After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells are incubated at 37±0.2.0° C. with 5.0±1.0% CO₂ and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies. Cells are incubated at 37±2.0° C. with 5.0±1.0% CO₂ and passaged every three to five days in the T-500 flask and every five to seven days in the ten layer cell stack (10CS). Cells should not remain in the T-500 flask for more than 10 days prior to passaging. Quality Control (QC) release testing for safety of the Bulk Drug Substance includes sterility and endotoxin testing. When cell confluence in the T-500 flask is .gtoreq.95%, cells are passaged to a 10 CS culture vessel. Alternately, two Five Layer Cell Stacks (5 CS) or a 10 Layer Cell Factory (10 CF) can be used in place of the 10 CS. 10CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37±2.0° C. with 5.0±1.0% CO₂ and fed with fresh Complete Growth Media every five to seven days. Cells should not remain in the 10 CS for more than 20 days prior to passaging. In one embodiment, the passaged dermal fibroblasts are rendered substantially free of immunogenic proteins present in the culture medium by incubating the expanded fibroblasts for a period of time in protein free medium, Primary Harvest When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

### III. Coronavirus

The disclosure concerns methods and compositions for treatment or prevention of at least any virus referred to herein.

Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and about 40 species. They are enveloped viruses having a positive-sense single-stranded RNA genome and a nucleocapsid having helical symmetry. The genome size of coronaviruses ranges from about 26-32 kilobases.

The present disclosure encompasses treatment or prevention of infection of any virus in the Coronaviridae family. In certain embodiments, the disclosure encompasses treatment or prevention of infection of any virus in the subfamily Coronavirinae and including the four genera, Alpha-, Beta-, Gamma-, and Deltacoronavirus. In specific embodiments, the disclosure encompasses treatment or prevention of infection of any virus in the genus of Betacoronavirus, including the subgenus Sarbecovirus and including the species of severe acute respiratory syndrome-related coronavirus. In specific embodiments, the disclosure encompasses treatment or prevention of infection of any virus in the species of severe acute respiratory syndrome-related coronavirus, including the strains severe acute respiratory syndrome coronavirus (SARS-CoV) and severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS), cluster, or sub-cluster of the species of severe acute respiratory syndrome-related coronavirus, including at least SARS-CoV-2. In specific embodiments, the virus being treated with methods and compositions of the disclosure is not SARS-CoV and is not MERS-CoV. In specific embodiments, the virus being treated with methods and compositions of the disclosure is SARS-CoV or is MERS-CoV. In specific embodiments, the virus has a genome length between about 29000 to about 30000, between about 29100 and 29900, between about 29200 and 29900, between about 29300 and 29900, between about 29400 and 29900, between about 29500 and 29900, between about 29600 and 29900, between about 29700 and 29900, between about 29800 and 29900, or between about 29780 and 29900 base pairs in length.

Examples of specific SARS-CoV-2 viruses include the following listed in the NCBI GenBank® Database, and these GenBank® Accession sequences are incorporated by reference herein in their entirety: (a) LC534419 and LC534418 and LC528233 and LC529905 (examples of different strains from Japan); (b) MT281577 and MT226610 and NC_045512 and MN996531 and MN908947 (examples of different strains from China); (c) MT281530 (Iran); (d) MT126808 (Brazil); (e) MT020781 (Finland); (f) MT093571 (Sweden); (g) MT263074 (Peru); (h) MT292582 and MT292581 and MT292580 and MT292579 (examples of different strains from Spain); (i) examples from the United States, such as MT276331 (TX); MT276330 (FL); MT276328 (OR) MT276327 (GA); MT276325 (WA); MT276324 (CA); MT276323 (RI); MT188341 (MN); and (j) MT276598 (Israel). In particular embodiments, the disclosure encompasses treatment or prevention of infection of any of these or similar viruses, including viruses whose genome has at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. In particular embodiments, the disclosure encompasses treatment or prevention of infection of any of these or similar viruses, including viruses whose genome has its entire sequence that is greater than 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. As one specific example, the present disclosure includes methods of treatment or prevention of infection of a virus having a genome sequence of SEQ ID NO:1 (represented by GenBank® Accession No. NC_045512; origin Wuhan, China) and any virus having a genome sequence with at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to SEQ ID NO:1. Infection with any strain of SARS-CoV-2 may be treated or prevented, including at least B.1.526, B.1.526.1, B.1.525, B.1.1.7, B.1.351, B.1.427, B.1.429, B.1.617, P.1, and P.2.

### V. Kits of the Disclosure

Any of the cellular and/or non-cellular compositions described herein or similar thereto may be comprised in a kit. In a non-limiting example, one or more reagents for use in methods for preparing cellular therapy may be comprised in a kit. Such reagents may include cells, one or more cytokines, one or more growth factors, carriers, media, enzymes, buffers, nucleotides, salts, primers, and so forth. The kit may comprise any compounds listed in the disclosure. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following examples are included to demonstrate particular embodiments of the disclosure. It should be appreciated by those of skill in the art that the techniques disclosed in the examples that follow represent techniques discovered by the inventor(s) to function well in the practice of the methods and compositions of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the disclosure.

### Example 1: Suppression of Monocyte and Endothelial Cells Tissue Factor Expression by Coculture with Fibroblasts

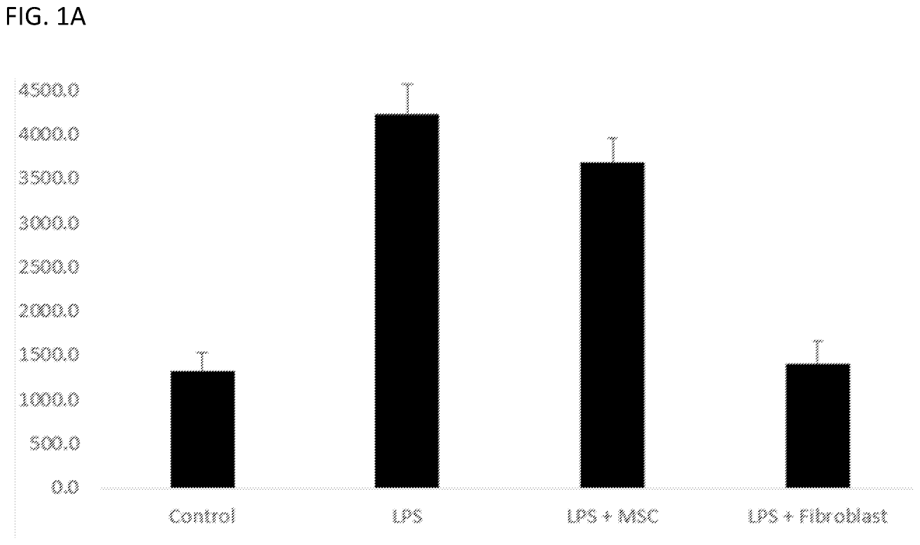

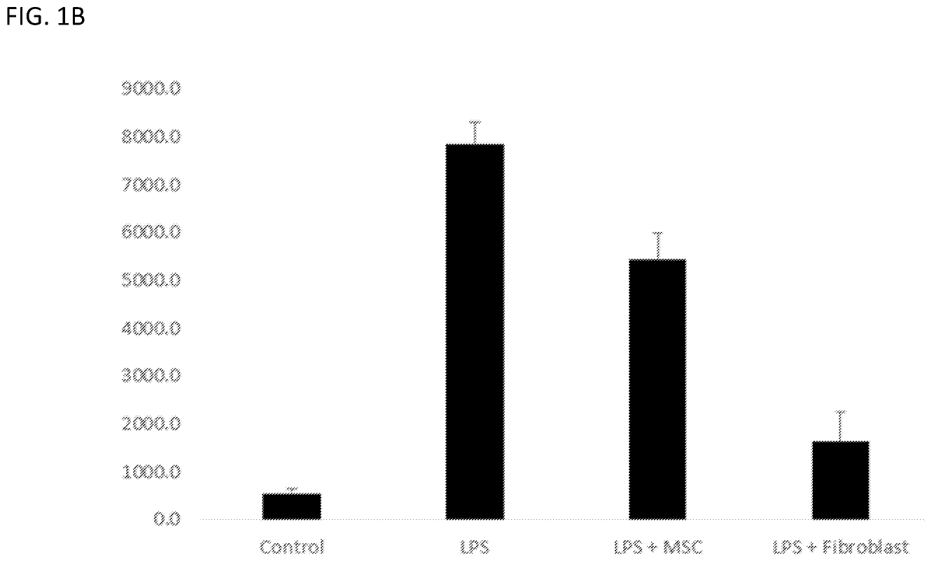

Foreskin fibroblasts were obtained from AllCells and cultured in Optimem media with 10% fetal calf serum at 5% carbon dioxide. Cells were plated at a one to one ratio with monocytes (FIG. 1A) or endothelial cells (FIG. 1B) (Human Umbilical Vein Endothelial Cells) and activated with lipopolysaccharide (LPS) at the 100 ng/ml. Expression of tissue factor was assessed after 48 hours of culture by flow cytometry and expressed as Mean Fluorescent Intensity (MFI). A significant decrease of tissue factor expression was observed when cells were cultured with mesenchymal stem cells as well as fibroblasts. Under these conditions, fibroblasts were superior to mesenchymal stem cells in reducing tissue factor expression.

It is known that under certain conditions fibroblasts are capable of producing interleukin-1 and/or other inflammatory cytokines. The invention teaches that fibroblasts may be treated be gene editing of IL-1 and/or other inflammatory mediators in order to prevent expression of inflammatory cytokines by fibroblasts after administration intradiscally. In some embodiments of the disclosure, TNF-alpha and inflammatory mediators are suppressed in the brain, however, fibroblasts may be pretreated with TNF-alpha in a manner to induce expression of growth factors and/or proliferation such as described in this following publication and incorporated by reference.

The present disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims. As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.” All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents. Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
1. Zhu N, Zhang D, Wang W, Li X, Yang B, Song J, Zhao X, Huang B, Shi W, Lu R et al: A Novel Coronavirus from Patients with Pneumonia in China, 2019. N Engl J Med 2020, 382(8):727-733.
2. Guo Y R, Cao Q D, Hong Z S, Tan Y Y, Chen S D, Jin H J, Tan K S, Wang D Y, Yan Y: The origin, transmission and clinical therapies on coronavirus disease 2019 (COVID-19) outbreak—an update on the status. Mil Med Res 2020, 7(1):11.
3. WHO WHO: Coronavirus disease (COVID-19) outbreak 2020:https://www.who.int/emergencies/diseases/novel-coronavirus-2019.
4. Zhang S, Diao M, Yu W, Pei L, Lin Z, Chen D: Estimation of the reproductive number of Novel Coronavirus (COVID-19) and the probable outbreak size on the Diamond Princess cruise ship: A data-driven analysis. Int J Infect Dis 2020.
5. Zhao S, Lin Q, Ran J, Musa S S, Yang G, Wang W, Lou Y, Gao D, Yang L, He D et al: Preliminary estimation of the basic reproduction number of novel coronavirus (2019-nCoV) in China, from 2019 to 2020: A data-driven analysis in the early phase of the outbreak. Int J Infect Dis 2020, 92:214-217.
6. Huang C, Wang Y, Li X, Ren L, Zhao J, Hu Y, Zhang L, Fan G, Xu J, Gu X et al: Clinical features of patients infected with 2019 novel coronavirus in Wuhan, China. Lancet 2020, 395(10223):497-506.
7. Wang D, Hu B, Hu C, Zhu F, Liu X, Zhang J, Wang B, Xiang H, Cheng Z, Xiong Y et al: Clinical Characteristics of 138 Hospitalized Patients With 2019 Novel Coronavirus-Infected Pneumonia in Wuhan, China. JAMA 2020.
8. Chen N, Zhou M, Dong X, Qu J, Gong F, Han Y, Qiu Y, Wang J, Liu Y, Wei Y et al: Epidemiological and clinical characteristics of 99 cases of 2019 novel coronavirus pneumonia in Wuhan, China: a descriptive study. Lancet 2020, 395(10223):507-513.
9. Grasselli G, Pesenti A, Cecconi M: Critical Care Utilization for the COVID-19 Outbreak in Lombardy, Italy: Early Experience and Forecast During an Emergency Response. JAMA 2020.
10. Wu C, Chen X, Cai Y, Xia J, Zhou X, Xu S, Huang H, Zhang L, Zhou X, Du C et al: Risk Factors Associated With Acute Respiratory Distress Syndrome and Death in Patients With Coronavirus Disease 2019 Pneumonia in Wuhan, China. JAMA Intern Med 2020.
11. Shi H, Han X, Jiang N, Cao Y, Alwalid O, Gu J, Fan Y, Zheng C: Radiological findings from 81 patients with COVID-19 pneumonia in Wuhan, China: a descriptive study. Lancet Infect Dis 2020.
12. Xu Z, Shi L, Wang Y, Zhang J, Huang L, Zhang C, Liu S, Zhao P, Liu H, Zhu L et al: Pathological findings of COVID-19 associated with acute respiratory distress syndrome. Lancet Respir Med 2020.
13. Tian S, Hu W, Niu L, Liu H, Xu H, Xiao S Y: Pulmonary pathology of early phase 2019 novel coronavirus (COVID-19) pneumonia in two patients with lung cancer. J Thorac Oncol 2020.
14. Gattinoni L, Chiumello D, Caironi P, Busana M, Romitti F, Brazzi L, Camporota L: COVID-19 pneumonia: different respiratory treatments for different phenotypes? Intensive Care Med 2020, 46(6):1099-1102.
15. Rello J, Storti E, Belliato M, Serrano R: Clinical phenotypes of SARS-CoV-2: implications for clinicians and researchers. Eur Respir J 2020, 55(5).
16. Gonzalez-Duarte A, Norcliffe-Kaufmann L: Is ‘happy hypoxia’ in COVID-19 a disorder of autonomic interoception? A hypothesis. Clin Auton Res 2020.
17. Tobin M J, Laghi F, Jubran A: Why COVID-19 Silent Hypoxemia is Baffling to Physicians. Am J Respir Crit Care Med 2020.
18. U R A, Verma K: Happy Hypoxemia in COVID-19-A Neural Hypothesis. ACS Chem Neurosci 2020, 11(13):1865-1867.
19. Couzin-Frankel J: The mystery of the pandemic's ‘happy hypoxia’. Science 2020, 368(6490):455-456.
20. Iba T, Levy J H, Levi M, Thachil J: Coagulopathy in COVID-19. J Thromb Haemost 2020.
21. Panigada M, Bottino N, Tagliabue P, Grasselli G, Novembrino C, Chantarangkul V, Pesenti A, Peyvandi F, Tripodi A: Hypercoagulability of COVID-19 patients in intensive care unit: A report of thromboelastography findings and other parameters of hemostasis. J Thromb Haemost 2020, 18(7):1738-1742.
22. Rivas-Pollmar M I, Alvarez-Roman M T, Butta-Coll N V, Martin Salces M, Garcia-Barcenilla S, Jimenez-Yuste V: Thromboprophylaxis in a patient with COVID-19 and severe hemophilia A on emicizumab prophylaxis. J Thromb Haemost 2020.
23. Iba T, Levy J H, Connors J M, Warkentin T E, Thachil J, Levi M: The unique characteristics of COVID-19 coagulopathy. Crit Care 2020, 24(1):360.
24. Luan Y Y, Liu Y, Liu X Y, Yu B J, Chen R L, Peng M, Ren D, Li H L, Huang L, Liu Y et al: Coronavirus disease 2019 (COVID-19) associated coagulopathy and its impact on outcomes in Shenzhen, China: A retrospective cohort study. Thromb Res 2020, 195:62-68.
25. Lodigiani C, lapichino G, Carenzo L, Cecconi M, Ferrazzi P, Sebastian T, Kucher N, Studt J D, Sacco C, Alexia B et al: Venous and arterial thromboembolic complications in COVID-19 patients admitted to an academic hospital in Milan, Italy. Thromb Res 2020, 191:9-14.
26. Miesbach W, Makris M: COVID-19: Coagulopathy, Risk of Thrombosis, and the Rationale for Anticoagulation. Clin Appl Thromb Hemost 2020, 26:1076029620938149.
27. FitzGerald E S, Jamieson A M: Unique transcriptional changes in coagulation cascade genes in SARS-CoV-2-infected lung epithelial cells: A potential factor in COVID-19 coagulopathies. bioRxiv 2020.
28. Ackermann M, Verleden S E, Kuehnel M, Haverich A, Welte T, Laenger F, Vanstapel A, Werlein C, Stark H, Tzankov A et al: Pulmonary Vascular Endothelialitis, Thrombosis, and Angiogenesis in Covid-19. N Engl J Med 2020, 383(2):120-128.
29. Jutila M A, Berg E L, Kishimoto T K, Picker L J, Bargatze R F, Bishop D K, Orosz C G, Wu N W, Butcher E C: Inflammation-induced endothelial cell adhesion to lymphocytes, neutrophils, and monocytes. Role of homing receptors and other adhesion molecules. Transplantation 1989, 48(5):727-731.
30. Swerlick R A, Lawley T J: Role of microvascular endothelial cells in inflammation. J Invest Dermatol 1993, 100(1):111S-115S.
31. Hayasaka H, Taniguchi K, Fukai S, Miyasaka M: Neogenesis and development of the high endothelial venules that mediate lymphocyte trafficking. Cancer Sci 2010, 101(11):2302-2308.
32. Mueller F J, Serobyan N, Schraufstatter I U, DiScipio R, Wakeman D, Loring J F, Snyder E Y, Khaldoyanidi S K: Adhesive interactions between human neural stem cells and inflamed human vascular endothelium are mediated by integrins. Stem Cells 2006, 24(11):2367-2372.
33. Lu M, Xue R, Wang P, Wang X, Tian X, Liu Y, Wang S, Cui A, Xie J, Le L et al: Induced pluripotent stem cells attenuate chronic allogeneic vasculopathy in an integrin beta-1-dependent manner. Am J Transplant 2020.
34. Henschler R, Deak E, Seifried E: Homing of Mesenchymal Stem Cells. Transfus Med Hemother 2008, 35(4):306-312.
35. Goshua G, Pine A B, Meizlish M L, Chang C H, Zhang H, Bahel P, Baluha A, Bar N, Bona R D, Burns A J et al: Endotheliopathy in COVID-19-associated coagulopathy: evidence from a single-centre, cross-sectional study. Lancet Haematol 2020.
36. Helms J, Tacquard C, Severac F, Leonard-Lorant I, Ohana M, Delabranche X, Merdji H, Clere-Jehl R, Schenck M, Fagot Gandet F et al: High risk of thrombosis in patients with severe SARS-CoV-2 infection: a multicenter prospective cohort study. Intensive Care Med 2020, 46(6):1089-1098.
37. Ladikou E E, Sivaloganathan H, Milne K M, Arter W E, Ramasamy R, Saad R, Stoneham S M, Philips B, Eziefula A C, Chevassut T: Von Willebrand factor (vWF): marker of endothelial damage and thrombotic risk in COVID-19? Clin Med (Lond) 2020.
38. Breakey N, Escher R: D-dimer and mortality in COVID-19: a self-fulfilling prophecy or a pathophysiological clue? Swiss Med Wkly 2020, 150:w20293.
39. Escher R, Breakey N, Lammle B: ADAMTS13 activity, von Willebrand factor, factor VIII and D-dimers in COVID-19 inpatients. Thromb Res 2020, 192:174-175.
40. Zachariah U, Nair S C, Goel A, Balasubramanian K A, Mackie I, Elias E, Eapen C E: Targeting raised von Willebrand factor levels and macrophage activation in severe COVID-19: Consider low volume plasma exchange and low dose steroid. Thromb Res 2020, 192:2.
41. Escher R, Breakey N, Lammle B: Severe COVID-19 infection associated with endothelial activation. Thromb Res 2020, 190:62.
42. Poissy J, Goutay J, Caplan M, Parmentier E, Duburcq T, Lassalle F, Jeanpierre E, Rauch A, Labreuche J, Susen S et al: Pulmonary Embolism in Patients With COVID-19: Awareness of an Increased Prevalence. Circulation 2020, 142(2):184-186.
43. Neri T, Nieri D, Celi A: P-selectin blockade in COVID-19-related ARDS. Am J Physiol Lung Cell Mol Physiol 2020, 318(6):L1237-L1238.
44. Monach P A: Biomarkers in vasculitis. Curr Opin Rheumatol 2014, 26(1):24-30.
45. Calabro P, Gragnano F, Golia E, Grove E L: von Willebrand Factor and Venous Thromboembolism: Pathogenic Link and Therapeutic Implications. Semin Thromb Hemost 2018, 44(3):249-260.
46. Peng X, Wang X, Fan M, Zhao J, Lin L, Liu J: Plasma levels of von Willebrand factor in type 2 diabetes patients with and without cardiovascular diseases: A meta-analysis. Diabetes Metab Res Rev 2020, 36(1):e3193.
47. Gragnano F, Sperlongano S, Golia E, Natale F, Bianchi R, Crisci M, Fimiani F, Pariggiano I, Diana V, Carbone A et al: The Role of von Willebrand Factor in Vascular Inflammation: From Pathogenesis to Targeted Therapy. Mediators Inflamm 2017, 2017:5620314.
48. Gupta A, Madhavan M V, Sehgal K, Nair N, Mahajan S, Sehrawat T S, Bikdeli B, Ahluwalia N, Ausiello J C, Wan E Y et al: Extrapulmonary manifestations of COVID-19. Nat Med 2020, 26(7):1017-1032.
49. Amraei R, Rahimi N: COVID-19, Renin-Angiotensin System and Endothelial Dysfunction. Cells 2020, 9(7).
50. Hernanz R, Martinez-Revelles S, Palacios R, Martin A, Cachofeiro V, Aguado A, Garcia-Redondo L, Barrus M T, de Batista P R, Briones A M et al: Toll-like receptor 4 contributes to vascular remodelling and endothelial dysfunction in angiotensin II-induced hypertension. Br J Pharmacol 2015, 172(12):3159-3176.
51. De Batista P R, Palacios R, Martin A, Hernanz R, Medici C T, Silva M A, Rossi E M, Aguado A, Vassallo D V, Salaices M et al: Toll-like receptor 4 upregulation by angiotensin II contributes to hypertension and vascular dysfunction through reactive oxygen species production. PLoS One 2014, 9(8):e104020.
52. Carrillo-Sepulveda M A, Spitler K, Pandey D, Berkowitz D E, Matsumoto T: Inhibition of TLR4 attenuates vascular dysfunction and oxidative stress in diabetic rats. J Mol Med (Berl) 2015, 93(12):1341-1354.
53. Montezano A C, Nguyen Dinh Cat A, Rios F J, Touyz R M: Angiotensin II and vascular injury. Curr Hypertens Rep 2014, 16(6):431.
54. Zhou Y, Little P J, Downey L, Afroz R, Wu Y, Ta H T, Xu S, Kamato D: The Role of Toll-like Receptors in Atherothrombotic Cardiovascular Disease. ACS Pharmacol Transl Sci 2020, 3(3):457-471.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of reducing coagulopathy or reducing the risk of coaguolopathy by administering to an individual in need thereof a therapeutically effective amount of fibroblasts, and/or modified fibroblasts, and/or derivatives of fibroblasts.

2. The method of claim 1, wherein said coagulopathy is associated with upregulated production of tissue factor in the individual.

3. The method of claim 2, wherein said tissue factor is expressed on endothelial cells.

4. The method of claim 2 or 3, wherein said tissue factor is expressed on monocytes.

5. The method of any one of claims 1-4, wherein said fibroblasts are derived from a tissue selected from the group consisting of a) placenta; b) skin; c) Wharton's Jelly; d) adipose; e) bone marrow; f) peripheral blood; g) cord blood; h) omentum; i) amniotic fluid; j) amniotic membrane; or k) a combination thereof.

6. The method of any one of claims 1-5, wherein said fibroblasts, and/or modified fibroblasts, and/or derivatives of fibroblasts are autologous, allogeneic, or xenogeneic with respect to the individual.

7. The method of any one of claims 1-6, wherein said coagulopathy is associated with reduction of one or more endothelial anticlotting factors.

8. The method of claim 7, wherein said endothelial anticlotting factor is Protein C.

9. The method of claim 7 or 8, wherein said endothelial anticlotting factor is thrombomodulin.

10. The method of claim 7, 8, or 9, wherein said endothelial anticlotting factor is anti-thrombin III.

11. The method of any one of claims 7-10, wherein said one or more anticlotting factors are expressed on endothelial cells.

12. The method of any one of claims 7-11, wherein said anticlotting factors are expressed on monocytes.

13. The method of any one of claims 1-12, wherein said coagulopathy is caused by or is associated with inflammation.

14. The method of claim 13, wherein said inflammation is associated with a 50% or more increase in plasma TNF-alpha concentration as compared to an age-matched healthy control subject.

15. The method of claim 13 or 14, wherein said inflammation is associated with a 50% or more increase in plasma interleukin-1 concentration as compared to an age-matched healthy control subject.

16. The method of any one of claims 13-15, wherein said inflammation is associated with a 50% or more increase in plasma interleukin-6 concentration as compared to an age-matched healthy control subject.

17. The method of any one of claims 13-16, wherein said inflammation is associated with a 50% or more increase in plasma interleukin-8 concentration as compared to an age-matched healthy control subject.

18. The method of any one of claims 13-17, wherein said inflammation is associated with a 50% or more increase in plasma interleukin-12 concentration as compared to an age-matched healthy control subject.

19. The method of any one of claims 13-18, wherein said inflammation is associated with a 50% or more increase in plasma interleukin-17 concentration as compared to an age-matched healthy control subject.

20. The method of any one of claims 13-19, wherein said inflammation is associated with a 50% or more increase in plasma interleukin-18 concentration as compared to an age-matched healthy control subject.

21. The method of any one of claims 13-20, wherein said inflammation is associated with a 50% or more increase in plasma interleukin-21 concentration as compared to an age-matched healthy control subject.

22. The method of any one of claims 13-21, wherein said inflammation is associated with a 50% or more increase in plasma interleukin-27 concentration as compared to an age-matched healthy control subject.

23. The method of any one of claims 13-22, wherein said inflammation is associated with a 50% or more increase in plasma interleukin-33 concentration as compared to an age-matched healthy control subject.

24. The method of any one of claims 13-23, wherein said inflammation is associated with a 50% or more increase in plasma interferon gamma concentration as compared to an age-matched healthy control subject.

25. The method of any one of claim 1-24, wherein said fibroblast proliferates at a rate of 14-21 hours per cell multiplication.

26. The method of any one of claims 1-25, wherein said fibroblasts secrete 0.1 pg-100 pg of interleukin 1 receptor antagonist per culture of 1 million fibroblasts on a 75% confluent surface.

27. The method of any one of claims 1-25, wherein said fibroblasts secrete 0.1 pg-10 pg of interleukin 1 receptor antagonist per culture of 1 million fibroblasts on a 75% confluent surface.

28. The method of any one of claims 1-27, wherein said fibroblasts secrete 1 pg-500 pg of FGF-1 per culture of 1 million fibroblasts on a 75% confluent surface.

29. The method of any one of claims 1-28, wherein said fibroblasts substantially decrease the ability of responding T cells to proliferate in a mixed lymphocyte reaction.

30. The method of claim 29, wherein said substantial decrease in proliferation constitutes a decrease of more than 20% as compared to a control mixed lymphocyte reaction in which fibroblasts are not added.

31. The method of any one of claims 1-30, wherein said fibroblasts are treated with an effective amount of hCG to augment immune modulatory activity.

32. The method of claim 31, wherein said hCG is administered to the fibroblasts at a concentration of 1 nano Molar to 1 micro Molar per 1 million fibroblasts.

33. The method of claim 33, wherein said hCG is administered to the fibroblasts at a concentration of 10 nano Molar to 100 nano Molar per 1 million fibroblasts.

34. The method of any one of claims 1-33, wherein said fibroblasts are treated with TNF-alpha for a sufficient length of time and concentration to allow said fibroblasts to decrease expression of tissue factor on monocytes and/or endothelial cells.

35. The method of claim 34, wherein said fibroblasts are treated with 0.1 pg to 20 ng of TNF-alpha per million fibroblast cells for the time period of at least one second.

36. The method of any one of claims 1-35, wherein the fibroblast derivative comprises conditioned media from fibroblast culture, microvesicles obtained from fibroblasts, fragments of fibroblasts, exosomes obtained from fibroblasts, apoptotic vesicles obtained from fibroblasts, or a combination thereof.

37. The method of any one of claims 1-36, wherein the fibroblasts are activated with one or more cytokines, one or more growth factors, or a mixture thereof.

38. The method of claim 37, wherein the cytokine is selected from the group consisting of IFN-gamma, TNF-alpha, interleukin(IL)-1, IL-6, IL-7, IL-8, IL-12, IL-15, IL-17, IL-33, and a combination thereof.

39. The method of claim 37 or 38, wherein the growth factor is selected from the group consisting of FGF-1, VEGF, and a combination thereof.
